(19) United States
(12) Patent Application Publication
(10) Pub. No.: US 2013/0138050 A1
(43) Pub. Date: May 30, 2013

(54) DRIVE MECHANISM WITH A LOW FRICTION COATING FOR A DRUG DELIVERY DEVICE

(71) Applicants: Michael Jugl, Frankfurt (DE); Günther Sendatzki, Frankfurt (DE); Axel Teucher, Frankfurt (DE)

(72) Inventors: Michael Jugl, Frankfurt (DE); Günther Sendatzki, Frankfurt (DE); Axel Teucher, Frankfurt (DE)

(73) Assignee: SANOFI-AVENTIS DEUTSCHLAND GMBH, Frankfurt am Main (DE)

(21) Appl. No.: 13/695,175

(22) PCT Filed: May 3, 2011
(86) PCT No.: PCT/EP2011/056994

§ 371 Date: Feb. 13, 2013

(30) Foreign Application Priority Data
May 4, 2010 (EP) 10161835.3

(51) Int. Cl. A61M 5/315

(52) U.S. Cl. CPC A61M 5/31513
USPC 604/207; 604/230

(57) ABSTRACT

The present invention relates to a drive mechanism for a drug delivery device, comprising a number of mechanically interacting functional components among which at least a piston rod is adapted to transfer thrust to a piston of a cartridge that contains a medicinal product to be dispensed, wherein at least one of the functional components at least in sections comprises a friction-reducing slide coating. In this way, a force level required for dispensing and/or setting of a dose can be effectively reduced.

### CROSS REFERENCE TO RELATED APPLICATIONS

The present application is a U.S. National Phase Application pursuant to 35 U.S.C. §371 of International Application No. PCT/EP2011/056994 filed May 3, 2011, which claims priority to European Patent Application No. 10161835.3 filed on May 4, 2010. The entire disclosure contents of these applications are herewith incorporated by reference into the present application.

### FIELD OF INVENTION

The present invention relates to a drug delivery device such as a pen-type injector and to a corresponding drive mechanism allowing to administer a single or a number of pre-set doses of a medicinal product. In particular, the invention further relates to functional components of a drive mechanism being mechanically engaged among themselves and which serve to apply well-defined thrust to a piston of a cartridge that contains the medicinal product to be dispensed by the drug delivery device.

### BACKGROUND

Drug delivery devices allowing for multiple dosing of a required dosage of a liquid medicinal product, such as liquid drugs, and further providing administration of the liquid to a patient, are as such well-known in the art. Generally, such devices have substantially the same purpose as that of an ordinary syringe.

Pen-type injectors of this kind have to meet a number of user specific requirements. For instance in case of those with diabetes, many users will be physically infirm and may also have impaired vision. Therefore, these devices need to be robust in construction, yet easy to use, both in terms of the manipulation of the parts and understanding by a user of its operation. Further, the dose setting must be easy and unambiguous and where the device is to be disposable rather than reusable, the device should be inexpensive to manufacture and easy to dispose. In order to meet these requirements, the number of parts and steps required to assemble the device and an overall number of material types the device is made from have to be kept to a minimum.

In particular with manually operated drive mechanisms of such drug delivery devices, a user may configure the drive mechanism in such a way, that a well-defined amount of medicinal product is dispensed during each dose dispensing procedure. Typically, a piston rod or a respective drive sleeve is axially guided in a housing of the drug delivery device or its drive mechanism, respectively. In this way axially and distally directed thrust can be transferred to a moveable piston of the cartridge, which in turn leads to a dispensing of a precise amount of the medicinal product.

In manually driven drug delivery devices and drive mechanisms, thrust to be transferred to the cartridge's piston is to be generated by the user himself Depending on the specific implementation of the drive mechanism, a dose button typically protruding from a distal end section of the drug delivery device may therefore have to be depressed in distal direction. Such user-initiated displacement of the dose button then transfers to a respective distally directed displacement of the piston rod, wherein the various functional components of the drive mechanism may become subject to linear, rotational and/or to a respective combined linear and rotational movement. Furthermore, the drive mechanism may also comprise respective clutch means, e.g. for selectively switching the drive mechanism in dose setting or dose dispensing mode.

Furthermore, there exist various drive mechanisms that provide automatic or semi-automatic dispensing of a liquid drug. Such drive mechanisms may either comprise energy storage means, such as springs, or may feature electrical drive means for generating a desired force- or thrust level in order to dispense a dose of the medicinal product.

Irrespective on whether the drive mechanism is manually, automatically or semi-automatically driven, the drive mechanism as well as its functional components being mechanically engaged, are typically subject to non-negligible friction. This applies particularly for threaded engagement of functional components, especially when implemented in a non-self-interlocking way, wherein for instance, a linearly directed movement of a functional component is transferred to a rotational movement of another functional component, or vice versa.

Due to inevitable mechanical friction among the functional components of drive mechanisms of such drug delivery devices, the force level to be applied for initiating of a dose dispensing or dose setting procedure unavoidably raises. This further implies a respective reinforcement of the functional component, typically leading to an increase in size and dimensions of respective functional components, the drive mechanism and hence to an increase in size of the drug delivery device itself.

Additionally, internal friction leads to motion-impeding actuation of the drive mechanism, irrespective on whether the mechanism is driven manually, automatically or semi-automatically.

It is therefore an object of the present invention, to provide a drive mechanism, a drug delivery device and functional components thereof that provide a smooth and smooth-running actuation as well as an improved general handling of such drug delivery devices.

In addition, the invention should be universally applicable to a large variety of different drive mechanisms and drug delivery devices. The invention should also be easily and universally implementable in existing drive mechanism designs and/or drug delivery devices.

### SUMMARY

The present invention relates to a drive mechanism for a drug delivery device. The drive mechanism comprises a number of mechanically interacting functional components among which at least a piston rod is adapted to transfer thrust to a piston of a cartridge. Said cartridge is to be arranged in a housing, in particular in a cartridge holder of the drug delivery device. The cartridge contains a medicinal product, which is to be dispensed during a single or during consecutive dose dispensing actions.

The cartridge is typically designed as vial or carpule. At a distally directed end section, the cartridge is sealed by means of a piercing element, such like a septum, which is to be pierced by a piercing element, e.g. an injection needle or a cannula. At its opposite, hence proximal end section, the cartridge comprises a piston moveably disposed therein. By exerting distally directed thrust to the piston, e.g. by means of a drive mechanism's piston rod, a well-defined amount of medicinal product can be expelled from the cartridge and can be administered to a user, typically to a patient.

In order to provide a smooth and smooth running handling of the drive mechanism, at least one of the functional components is at least in sections provided with a friction-reducing coating. Hence, the at least one functional component at least in sections comprises a friction-reducing slide coating. In this way, internal friction among the functional components can be remarkably reduced. In effect, actuation forces to be externally applied to the drive mechanism or to be generated by the drive mechanism itself can be reduced, which is beneficial for the general handling and design of the device.

Additionally, mechanical stress arising during a dose dispensing action may also reduce, which allows for reducing the dimensions of the functional components. Consequently, the drive mechanism can become subject to a size- and mechanical stress-reduced design, generally even allowing to reduce overall weight and dimensions of the drive mechanism and/or of the drug delivery device.

In addition to the benefits regarding a reduction of forces to be applied, by having a friction-reducing coating, additional friction-reducing means, such like grease or comparable lubricants may become superfluous. Hence, the entire device becomes less prone to a contamination by lubricants even under conditions of a long-term use.

According to a preferred embodiment of the invention, the coating comprises at least one fluorocarbon component. In particular, the coating comprises polytetrafluoroethylene (PTFE) and/or fluorinated ethylene propylene (FEP). By providing the at least one functional component with a PTFE- or FEP-coating, a beneficial friction-reducing effect can be achieved.

Moreover, in practice it has turned out, that PTFE- and/or FEP-coatings provide a sufficient stability and robustness even under conditions of long-term use of the device. Tests have revealed, that such coatings remain substantially stable and do not scratch off during intended use of the drug delivery device.

In another aspect of the invention, the coating comprises a coefficient of static friction between 0.1 and 0.3, preferably between 0.12 and 0.20.

In a further and/or additional another embodiment, the coating may also comprise a coefficient of dynamic friction between 0.06 and 0.4, preferably between 0.08 and 0.3, most preferably, between 0.12 and 0.25. By realizing such static and/or dynamic friction coefficients, forces to be externally applied to the drive mechanism in order to move a piston rod in a desired direction can be reduced by 40% compared to identical drive mechanisms that lack a comparable friction-reducing coating.

According to another preferred embodiment, the coating comprises a thickness between 1 μm and 10 μm. By implementing a coating with a thickness of less than 10 μm, the invention becomes universally applicable to almost any existing drive mechanism. Hence, the gain in size of a functional component due to the coating is almost of negligible impact to the mechanical engagement of the functional components.

In another preferred embodiment, the functional component itself comprises a PTFE- or FEP-doped thermoplastic material. The functional components of the drive mechanism are preferably designed as injection-molded thermoplastics. In this way, the functional components can be manufactured dimensionally stable in large numbers at comparatively low costs.

By having a PTFE- or FEP-doped thermoplastic material as base material for the functional components, even in cases when the friction-reducing slide coating should be scrapped off, the respective dopants may still provide a sufficient friction-reducing effect.

In a further preferred embodiment, the coated functional component comprises polybutylene terephthalate (PBT). It is even conceivable, that the functional component consists of PBT coated with PTFE and/or FEP.

Usage of PTFE doped PBT is beneficial in that this material implies with national and international registry regulatory requirements in terms of biocompatibility, which generally allows to make use of such materials in medical devices.

In a further preferred embodiment, the thermoplastic material of the at least one coated functional component comprises a temperature stability of at least 160° C. Preferably, the thermoplastic material comprises a temperature stability of up to 200° C., or even of up to 220° C.

Such temperature stability is beneficial and may be required for the process of coating the thermoplastic material, e.g. with PTFE and/or FEP. Coating of the at least one functional component with at least one fluorocarbon requires a curing process, which has to take place at a certain temperature level, typically in the range of 160° C. and above.

Therefore, it is beneficial, that the base material of said functional component remains chemically and mechanically stable during the entire coating process.

According to further preferred embodiments, the at least one and the at least partially coated functional component can be configured as a piston rod being moveably disposed in a housing. The functional component may be further configured as an insert or a receptacle threadedly engaged or keyed engaged with the piston rod, wherein the insert serves to radially fix the piston rod within the housing. For instance, the insert may be configured as a lead screw threadedly engaged with the piston rod.

In another alternative, the friction-reduced functional component can be configured as a drive sleeve being threadedly engaged with the piston rod and/or with other functional components of the drive mechanism. Furthermore, it is conceivable, to configure the at least one friction-reduced functional component as a dose dial sleeve, as a dose dial button and/or as a clutch means or components therefore for operably and/or mechanically interconnecting the components of the drive mechanism as mentioned above.

The invention is by no way restricted to particular or selected components of the drive mechanism. Therefore, generally all components of a drive mechanism of the drug delivery device can be at least partially or even entirely provided with a friction-reducing coating. Alternatively, depending on the general design and construction of a drive mechanism, it may already be sufficient to provide only one or a few selective functional components of the drive mechanism with a friction-reducing slide coating, such as e.g. the piston rod.

In another independent aspect, the invention provides a drug delivery device for dispensing of a dose of a medicinal product. The drug delivery device comprises a cartridge holder, which is adapted to receive a cartridge that has a piston slidably disposed therein. Furthermore, the drug delivery device comprises a drive mechanism according to the present invention, which at least comprises a piston rod being adapted to exert thrust to the piston of the cartridge.

In another independent aspect, the invention further provides a functional component of a drive mechanism of a drug delivery device, which comprises a thermoplastic component as core or base material and which is at least partially coated with a friction-reducing slide coating. Here, the coating comprises polytetrafluoroethylene (PTFE) and/or fluorinated ethylene propylene (FEP).

Furthermore, the core or base material comprises PTFE- or FEP-doped thermoplastic material. In particular, either one or several of the components: piston rod, insert, receptacles, drive sleeve, dose dial sleeve, dose dial pattern and/or clutch means can be designed as functional component being entirely or at least partially provided with a friction-reducing slide coating.

### BRIEF DESCRIPTION OF THE DRAWINGS

In the following, an embodiment of a drug delivery device according to the present invention is exemplary described by making reference to the drawings, in which:

FIG. 1 shows a drug delivery device in cross section in an initial configuration and

FIG. 2 illustrates the drug delivery device according to FIG. 1 prior dose dispensing.

### DETAILED DESCRIPTION

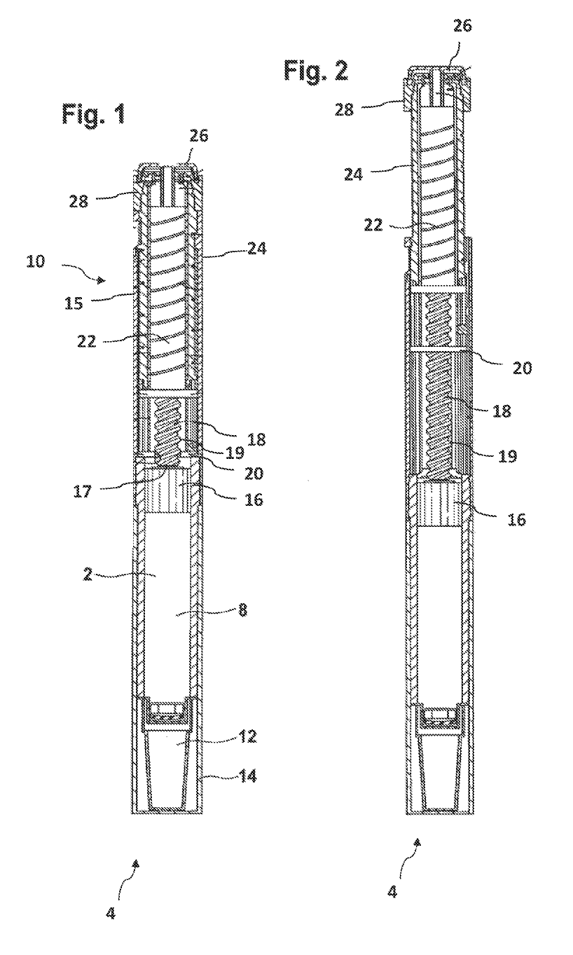

The drug delivery device 4 as illustrated in FIGS. 1 and 2 comprises a cartridge holder 2 that serves to house and to receive a cartridge 8 filled with a medicinal product to be dispensed by the drive mechanism 10 of the drug delivery device 4. The cartridge 8 comprises at its upper, hence proximal, end section a piston 16 moveably disposed in said cartridge 8. A removable cap 12 is releasably retained at a lower, distal end of the cartridge holder 2. In use, said cap 12 can be replaced by a suitable piercing element, such an injection needle, cannula or the like for dispensing and administering the liquid drug to a patient.

The entire cartridge holder 2 is further covered by another replaceable cap 14. Preferably, the outer dimensions of said replaceable cap 14 are similar or identical to the outer dimensions of a main housing component 15, which serves to accommodate the drive mechanism 10.

The drive mechanism 10 comprises a piston rod 18 that has an outer thread 19 matching with an inner thread of an axially displaceable insert or lead screw 20. Moreover, the piston rod 18 is also threadedly engaged with an inner thread of an axially displaceable drive sleeve 22. Said piston rod 18 comprises a second threaded portion at its upper, proximal end section, which is not explicitly illustrated in the Figures. With its second threaded portion, it is threadedly engaged with the inner thread of the drive sleeve 22.

The piston rod 18 comprises a pressure piece 17 at its lower, hence distal, end section, which buts against a proximal end face of the piston 16 of the cartridge 8. In this way, distally directed thrust provided by the piston rod 18 is transferred to a respective distally directed movement of the piston 16, thereby expelling a pre-defined amount of the liquid medicinal product contained in the cartridge 8.

Preferably, first and second threads of the piston rod 18 are oppositely directed and comprise different leads. In this way, an axial displacement of the drive sleeve 22 leads to a rotational movement of the piston rod, which due to the threaded engagement with the insert 20 becomes also subject to a respective axial displacement in distal direction, hence, towards the lower part of the drug delivery device 4.

As further illustrated in FIGS. 1 and 2, the drive mechanism 10 further comprises a dose dial sleeve 24 as well as a dose dial button 28, by means of which the drive mechanism 10 can be transferred into a configuration as illustrated in FIG. 2, wherein the drive sleeve 22 and the dose dial sleeve 24 together with the dose dial button 28 and a dose button 26 axially protrude from the housing 15 of the drive mechanism 10.

Starting from the configuration as illustrated in FIG. 2, a user may manually exert distally directed thrust to the dose button 26, which consequently leads to an axially and distally directed displacement of the entire drive mechanism 10. Due to the threaded engagement of the piston rod 18 with both, the drive sleeve 22 and the insert 20, distally directed movement of the piston rod 18 is reduced compared to the distally directed displacement of the drive sleeve 22.

According to the present invention, at least one component 18, 20, 22, 24 26, 28 of the drive mechanism 10 is provided with a friction-reducing slide coating for reducing activation and manipulation forces to be applied to the drug delivery device 4 and/or of its drive mechanism 10 for setting and/or for dispensing of a dose of the medicinal product.

The illustrated embodiment according to FIGS. 1 and 2 is only exemplary for the present invention. The drive mechanism 10 as illustrated in FIGS. 1 and 2 substantially corresponds to a drive mechanism as illustrated in WO 2004/078241 A1, which in its entirety is incorporated herein by reference.

Besides the illustrated embodiment, any other commercially available drive mechanism may become subject to the present invention by selectively providing at least one of its functional components with a friction-reducing slide coating according to the present invention.

1-13. (canceled)

14. Drive mechanism for a drug delivery device, comprising a number of mechanically interacting functional components among which at least a piston rod is adapted to transfer thrust to a piston of a cartridge that contains a medicinal product to be dispensed, wherein at least one of the functional components at least in sections comprises a friction-reducing slide coating.

15. The drive mechanism according to claim 14, wherein the coating comprises at least one fluorocarbon.

16. The drive mechanism according to claim 14, wherein the coating comprises polytetrafluoroethylene (PTFE) and/or fluorinated ethylene propylene (FEP).

17. The drive mechanism according to claim 14, wherein the coating comprises a coefficient of static friction between 0.1 and 0.3, preferably between 0.12 and 0.20.

18. The drive mechanism according to claim 14, wherein the coating comprises a coefficient of dynamic friction between 0.06 and 0.4, preferably between 0.08 and 0.3, most preferably between 0.12 and 0.25.

19. The drive mechanism according to claim 14, wherein the coating comprises a thickness between 1 μ and 10 μm.

20. The drive mechanism according to claim 14, wherein the functional component comprises a PTFE- or FEP-doped thermoplastic material.

21. The drive mechanism according to claim 20, wherein the thermoplastic material comprises polybutylene terephthalate (PBT).

22. The drive mechanism according to claim 20, wherein the thermoplastic material comprises a temperature stability of at least 160° C.

23. The drive mechanism according to claim 14, wherein the at least one at least partially coated functional component is configured:
as a piston rod movably disposed in a housing;
as an insert threadedly engaged or keyed engaged with the piston rod;
as a drive sleeve threadedly engaged with the piston rod;
as a dose dial sleeve;
as a dose dial button; and/or
as a clutch means for operably interconnecting said components.

24. Drug delivery device for dispensing of a dose of a medicinal product comprising:
a cartridge holder adapted to receive a cartridge having a slidably disposed piston therein and
a drive mechanism according to any one of the preceding claims, having a piston rod being adapted to exert thrust to the piston.

25. A functional component of a drive mechanism of a drug delivery device comprising a thermoplastic component as core material being at least partially coated with a friction-reducing slide coating.

26. The functional component according to claim 25, wherein the coating comprises polytetrafluoroethylene (PTFE) and/or fluorinated ethylene propylene (FEP) and wherein the core material comprises a PTFE- or FEP-doped thermoplastic material.
